(19) United States
(12) Patent Application Publication
(10) Pub. No.: US 2004/0193144 A1
(43) Pub. Date: Sep. 30, 2004

(54) Drug delivery device

(76) Inventor: John Krumme, Tahoe City, CA (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/470,251

(22) PCT Filed: Jan. 25, 2002
(86) PCT No.: PCT/IB02/00266

(30) Foreign Application Priority Data
Jan. 27, 2001 (GB) 0102171.6

(51) Int. Cl. A61K 9/22

(52) U.S. Cl. 604/890.1

(57) ABSTRACT

A drug delivery device which can be implanted in a patient, includes a reservoir for the drug with at least one dis-charge outlet through which the drug can be discharged. A drive member comprising a shape memory alloy is arranged in the device in a deformed configuration to act against the reservoir directly or indirectly by recovering from its deformed configuration by virtue of its elastic properties to cause the volume of the reservoir available for the drug to be reduced and to cause drug in the reservoir to be discharged from the reservoir. A flow controller for controlling the flow of the drug through the discharge outlet comprises a close-packed array of elongate rod members extending generally in the direction in which the drug flows out of the reservoir.

This invention relates to a drug delivery device which is suitable for implantation within a body to deliver controlled quantities of a drug within the body.

It can be desirable to delivery controlled quantities of a drug to a site within a human or animal body over a long period. This can be desired for example in the treatment of malignant tumours where a drug might be delivered to the site of the tumour. It can be important in urology in the control of incontinence and for the control of diabetes. It might also be useful for some patients in the treatment of arterial disease. Another application can be in the control of localised pain such as following surgery.

Techniques for controlled delivery of drugs from external reservoirs are well established. These generally involve supply of a drug through a catheter in which the drug is transported intravenously to the patient, and then systematically to the selected site. Control over the supply of the drug is achieved by means of appropriate valve components which are located externally of the patient. A disadvantage of such external drug delivery techniques is that the patient must be connected to the external reservoir by means of the catheter with its associated valve components, and the drug is delivered to the venous system rather than specifically to the appropriate local site.

The present invention provides a drug delivery device which can be implanted in a patient, which includes a reservoir for the drug, having at least one discharge outlet through which the drug can be discharged, a drive member which is arranged in the device in a deformed configuration to act against the reservoir directly or indirectly by recovering from its deformed configuration by virtue of its elastic properties to cause the volume of the reservoir available for the drug to be reduced and to cause drug in the reservoir to be discharged from the reservoir, and a flow controller which controls flow of the drug through the discharge outlet, comprising a close-packed array of elongate rod members extending generally in the direction in which the drug flows out of the reservoir.

In one aspect, the invention provides a drug delivery device which comprises:

a. a reservoir for the drug, having a discharge outlet towards one end thereof through which the drug can be discharged,

b. a drive member which is arranged in the device to act against the reservoir directly or indirectly to cause the volume of the reservoir available for the drug to be reduced and to cause drug in the reservoir to be discharged from the reservoir at the end in which the discharge outlet is located,

c. a flow controller which controls flow of the drug through the discharge outlet, the flow controller comprising a close-packed array of elongate rod members extending generally in the direction in which the drug flows out of the reservoir.

The rod members define at least one flow channels between the rod members and the wall of the reservoir for the drug to flow along. The resistance to flow of the drug through the flow controller (and therefore the rate at which drug is discharged from the device) is affected by factors such as the length of the rods, the cross-sectional area of the rods (their diameter when the rods have a circular cross-section), the number of rods (and therefore the number of flow channels that they define between them), the surface finish of the rods, and the viscosity of the drug to be discharged (which will often be insensitive to fluctuations in temperature when the device is in use because of the constant temperature environment within the body).

Preferably, the rod members are straight and are arranged in the device so that they extend substantially parallel to the general direction in which the drug flows out of the reservoir. However, other configurations of rod members might be used. For example, at least some of the rod members might have a helical configuration, with the result that the flow paths which are defined between the rod members are longer than if the rod members are straight. For example, a flow controller might include a plurality of rod members which are collectively twisted in the manner of the strands of a wire rope. When rod members are arranged in layers (for example with one or more layers of strands around a central core strand), the strands in each layer might follow helical paths on the surface of the underlying layer (or core).

The flow controller will often itself be straight, whether the rod members within it are themselves straight or helical. This has the advantage that the flow controller can be fitted conveniently in a common housing with other components of the delivery device such as a reservoir for the drug and a driver member. The flow controller can have a non-straight configuration, for example helical (preferably with a constant cross-section over at least a significant portion of its length), in which the rod members are all follow the helical configuration of the flow controller, preferably with a constant spatial relationship between them. It can be appropriate in some constructions for the flow controller to be located outside a housing for other components of the device, for example the driver member or the reservoir for the drug or both. This can be particularly appropriate when the flow controller is non-straight, for example helical. In this case, the outlet from the reservoir can be connected to the flow controller (directly or indirectly).

The flow controller can include an outer tube for retaining the rod members in a desired arrangement. When the flow controller is straight, the outer tube can be a housing tube for the delivery device. However, it can be appropriate for some constructions for the flow controller to include an outer tube for the rod members. This can be particularly appropriate when the flow controller is not straight, for example helical.

Examples of materials for the rod members include metallic materials such as certain stainless steels and titanium alloys, polymeric materials such as polytetrafluoroethylene, and ceramic materials such as certain quartz materials and other glasses. The material will be selected to be compatible with materials with which it will come into contact when the device is in use. Suitable materials should be capable of manufacture to controlled dimensions which are stable. This means that the device can be made to provide a suitable flow rate which is stable over a period in which the device is in use.

Examples of arrays of the rod members include three members in a triangular array, and four members in a square array. Preferred arrangements comprise a central rod member with at least one ring of rod members arranged around the central rod member. If the rods in a first ring have the same cross-sectional size as the central rod member, the number in the first circular array will be six. One or more additional circular arrays can be provided.

Preferably, the cross-section size (diameter when the rods have a circular cross-section) of each rod member in any one layer is substantially the same. It will often be preferred for all of the rod members of the flow member have substantially the same cross-section size.

Channels defined by an array of rods can be blocked selectively to provide a reduced controlled rate of flow of drug. The channels can be blocked partially using inserted rods, or by means of a flexible material which is forced into the channels.

Preferably, the arrangement of the rod members is stable so that the relative positions of the rod members does not change significantly when they are subjected to an inwardly directed force. This can be achieved conveniently when there are three rod members arranged with their centres at the apices of an equilateral triangle, or seven rod members with six arranged around one central rod member.

Preferably, the rod members are retained in the reservoir by engaging the inner wall of the reservoir towards the end in which the discharge outlet is located. The rod members can be pressed into the reservoir. When the reservoir is made from a material which can be made to shrink (for example, on application of heat), the reservoir can be made to shrink onto the rod members.

The engagement of the rod members within the reservoir can retain the rod members in a desired configuration. For many stable configurations of rod members, a reservoir with a circular cross-section will be suitable to retain the rod members. For example, this will apply when there are three rod members arranged with their centres at the apices of an equilateral triangle, or seven rod members with six arranged around one central rod member. A non-circular reservoir can help to retain other arrangements of rod members in a desired configuration. For example, a reservoir with a square cross-section can be useful when there are four rod members.

Preferably, the drive member comprises a shape memory alloy which has been treated so that it exhibits enhanced elastic properties, which is arranged in the device in a compressed configuration to act against the reservoir directly or indirectly by recovering from its compressed configuration by virtue of its elastic properties to cause the volume of the reservoir available for the drug to be reduced and to cause drug in the reservoir to be discharged from the reservoir at the end in which the discharge outlet is located.

This device has the advantage that, by appropriate treatment to give it enhanced elastic properties, the drive member can exert a controlled discharge force against reservoir to cause a large change in the volume of the reservoir. This enables the rate of discharge of the drug from within the reservoir to be controlled. The enhanced elastic properties of a shape memory alloy drive member, which can provide a large recoverable strain, means that the size of the drive member can be maintained small for a given range of displacement. This can enable the proportion of the volume of the reservoir in a small device to be maximised. It also enables the size of a device for delivery of a particular volume of drug to be minimised.

Enhanced elastic properties are available from shape memory alloys as a result of a transformation between martensite and austenite phases of the alloys, which make them particularly well suited to this application. The nature of the superelastic transformations of shape memory alloys is discussed in “Engineering Aspects of Shape Memory Alloys”, T W Duerig et al, on page 370, Butterworth-Heinemann (1990). Subject matter disclosed in that document is incorporated in this specification by this reference to the document. A principal transformation of shape memory alloys involves an initial increase in strain, approximately linearly with stress. This behaviour is reversible, and corresponds to conventional elastic deformation. Subsequent increases in strain are accompanied by relatively small increases in stress, over a limited range of strain to the end of the “loading plateau”. The loading plateau stress is defined by the positive inflection point on the loading portion of the stress/strain graph. Subsequent increases in strain are accompanied by larger increases in stress. On unloading, there is a decline in stress with reducing strain to the start of the “unloading plateau” evidenced by the existence of an inflection point along which stress changes little with reducing strain. At the end of the unloading plateau, stress reduces with approximately linear reducing strain. The unloading plateau stress is also defined by the inflection point on the stress/strain graph. Any residual strain after unloading to zero stress is either largely recoverable on heating or permanent set of the sample. Characteristics of this deformation, the loading plateau, the unloading plateau, the elastic modulus, the plateau length and the permanent set (defined with respect to a specific total deformation) are established, and are defined in, for example, “Engineering Aspects of Shape Memory Alloys”, on page 376.

Non-linear superelastic properties can be introduced in a shape memory alloy by a process which involves cold working the alloy for example by a process that involves pressing, swaging or drawing. The cold working step can be followed by an annealing step while the component is restrained in the configuration, resulting from the cold working step at a temperature that is sufficiently high to cause dislocations introduced by the cold working to combine, resulting in a more uniform dislocation distribution. This process can ensure that the deformation introduced by the cold work is retained.

Suitable shape memory alloys for use in the device of the invention include binary alloys, such as those in which the nickel content is at least about 50 at. %, preferably at least about 50.5 at. %. The nickel content will usefully be less than about 52 at. %, preferably less than about 51 at. %. The device can be formed from other Ni—Ti based alloys, including alloys with ternary and quaternary additions. Examples of elements that can be incorporated in the alloy include Fe, Co, Cr, Al, Cu and V. Added elements can be present in amounts up to about 10 at. %, preferably up to about 5 at. %.

The device of the invention has the advantage that, by using a shape memory alloy in the drive member which exhibits enhanced elastic properties, a controlled closing force can be exerted on the reservoir, the force being capable of being controlled over a range of movement. For example, when the alloy has been treated so that the element exhibits non-linear enhanced elastic behaviour (in which the stress/strain behaviour exhibits a plateau on loading and unloading, defined by points of inflection on a stress/strain graph) over the appropriate temperature range, the force exerted on the reservoir can be approximately constant throughout the range of displacement of the drive member. Significantly, the invention allows the force exerted on a reservoir to be adjusted by selection of a drive member which has appropriate properties by virtue of the treatment to which the shape memory alloy has been subjected to give it its enhanced elastic properties, so that the rate of discharge of the drug is appropriate having regard to the requirements for treatment of the patient. Furthermore, the control over the discharge of the drug from the implanted device is available without any need for connection to external control equipment.

The structure of the drive member will enable it to exert an appropriate force and an appropriate range of displacement. A suitable drive member might have a helical configuration, at least in part, for example in the form of a helical spring. Such a drive member might be provided by a generally flat strip, or a round wire, of a shape memory alloy which has been formed into the drive member by winding.

Especially when the drive member has a helical configuration, it can be located towards the end of the device that is opposite to the discharge outlet, the drive member recovering by expanding longitudinally from a compressed configuration.

The device of the invention can include a discharge piston which is acted on by the drive member, directly or indirectly, to cause it to move within the device towards the end in which the discharge outlet is located. The device can include a hollow tube which contains the reservoir and the discharge piston which is arranged to slide therein. The drive member can be contained within the hollow tube, and the device can include a retainer cap on the hollow tube at the end remote from the discharge outlet, for retaining the drive member within the tube.

Preferably, the drive member is positioned around the reservoir and recovers inwardly towards the reservoir from an expanded configuration. A suitable drive member has a lattice structure which comprises a network of interconnecting limbs defining apertures between them. The configuration of the drive member can change to cause the discharge piston to move by bending the limbs, which causes the shape of the apertures to change.

Techniques for making drive members of this kind from shape memory alloy materials are known. Such techniques can be used to make products such as cardiovascular stents. Generally, the drive member will be made from a tube of the alloy, by removing material by an operation which involves cutting, melting or vaporising the material. It is particularly preferred that the drive member by made by a laser cutting technique, but other cutting techniques might include stamping, cutting, and etching (especially photoetching). Known laser cutting techniques which can be used in this way include the use of a YAG laser.

The reservoir can be provided by a tubular member which can be cut to length to provide a reservoir of a desired volume. The hollow tube can define or contain the reservoir, and a piston member can be arranged to slide within the tube. Especially (but not exclusively) when the reservoir is provided by a tube in this way, it will be preferred for the device to include a retainer cap on the hollow tube at the end remote from the discharge outlet, for retaining the drive member within the tube. It can be preferable for a retainer cap to have at least one opening extending through it, especially when the device is formed from a tubular member which defines or contains the reservoir and in which the piston member slides. The opening can allow fluid to pass into the tubular member as the piston member slides to discharge the drug, so as to avoid creating a region of localised reduced pressure when the piston is moved to cause drug to be discharged.

The device can be made from a tube of a polymeric material. The material should be compatible with materials with which it will come into contact when the device is in use. It should be capable of withstanding forces to which it is subjected in use without deforming undesirably. For example, in the region in which the flow controller is located, it should not deform outwardly which could have the result of opening the channels for drug to flow past the flow controller and therefore of increasing the rate at which drug is discharged from the device. In the region of the reservoir, it should be dimensionally stable so that the volume of the reservoir does not increase when the piston member moves to urge the drug towards the discharge outlet. Preferably, the material should exhibit a low coefficient of friction, for example with respect to a delivery catheter. Suitable polymeric materials for the tubular member might include, for example, polymers of halogenated olefins, especially PTFE, polyesters, polyamides and polycarbonates. The material might be reinforced to give it the desired mechanical properties, for example using embedded fibres. This can have the advantage of allowing the device to be made from tube which has a small wall thickness while still being able to withstand forces to which it is subjected when in use.

Other materials for the tube might include, for example, certain metals such as certain stainless steels or titanium alloys.

Preferably, the discharge piston includes a plug member to seal the reservoir at the end thereof against which the drive member acts. It can be preferred for the plug member to include a gasket formed from a resilient material, in sliding contact with the wall of the reservoir. A plug member can slide along the reservoir, as a result of force exerted on it by the drive member, to cause drug within the reservoir to be urged towards the discharge outlet. The plug member can prevent, or at least minimise, leakage of the drug from the reservoir towards the end of the reservoir which is opposite to the discharge outlet.

The reservoir can include a collapsible bladder for containing the drug. The bladder will have an opening, usually at the end of the reservoir adjacent to the discharge outlet, through which drug within the bladder can be discharged from the bladder. The bladder can be made to collapse by the action against it of the discharge piston. When a bladder is included in the reservoir, it is possible for the plug member to be omitted from the discharge piston.

In order to prevent or at least to minimise loss of drug from the device, it can be preferred for the device to include a cap which can be fitted over the discharge outlet. The cap can be made from a dissolvable material, especially so that the cap will dissolve in body fluids after the device has been implanted. Examples of suitable materials for the cap include certain gelatins. When the rate of flow of drug out of the device is small, the device can be implanted without a cap with only a small risk of loss of drug.

It can be preferred for the device of the invention to assembled before drug is introduced into the reservoir. In order to permit drug to be introduced into the assembled device, it can be preferred for at least one of the rod members to comprise a hollow tube and a plug which can be received in the tube to seal it against fluid flow. Preferably, the plug is elongate, for example in the form of a narrow rod or length of wire.

The device of the invention can be used to deliver a drug to a predetermined site at a low dosage rate over an extended period. For example, by suitable selection of drive member and flow controller, flow rate of drug of less than about 1 ml per month can be achieved. The low flow rate that can be achieved means that the reservoir-for the drug can be kept small while still allowing sufficient drug to be carried within the reservoir for administration to the patient over an extended period. The use of drive member which consists at least in part of a shape memory alloy allows a significant displacement to be obtained from a compact drive member.

Accordingly, the device of the invention can be made with a transverse dimension (diameter when the device has a circular cross-section) of not more than about 2 mm, especially not more than about 1 mm. The use of a device which has a small transverse dimension has the advantage that the device can conveniently be implanted using apparatus of the kind which is know for delivery of drugs or of medical hardware to a desired location, for example comprising one or both of a needle and a catheter. Such apparatus can be used to introduce the device into a blood vessel, and to be delivered within the blood vessel to the desired location, or can be used for direct placement of the device in tissue or an organ to which the drug is to be supplied.

In a further aspect, the invention therefore provides a drug delivery assembly which comprises a device of the kind discussed above, and an applicator for implanting the device in a body, the applicator comprising at least one of (a) a needle which has a bore extending at least part way along its length which is open at one end, in which the device can be fitted, and (b) a catheter having a bore extending at least part way along its length, in which the device can slide along the catheter.

Preferably, the device of the invention includes a retrieval line which is attached to the device and which can extend from the device towards an extraction site, and which can be used to apply force to the device to retrieve it from the location at which drug is discharged towards the extraction site. Such a feature can be particularly useful when the device is implanted using a catheter. It can be preferred for the retrieval line to be formed from a shape memory alloy which has been treated so that it exhibits enhanced elastic properties. The retrieval line can then be used as a guide wire to steer a catheter to the desired location for discharge of the drug, for example in the manner disclosed in EP-A-141006. It can be convenient for the retrieval line and a shape memory alloy drive member to be formed as a single body.

Embodiments of the present invention will now be described by way of example with reference to the accompanying drawings, in which:

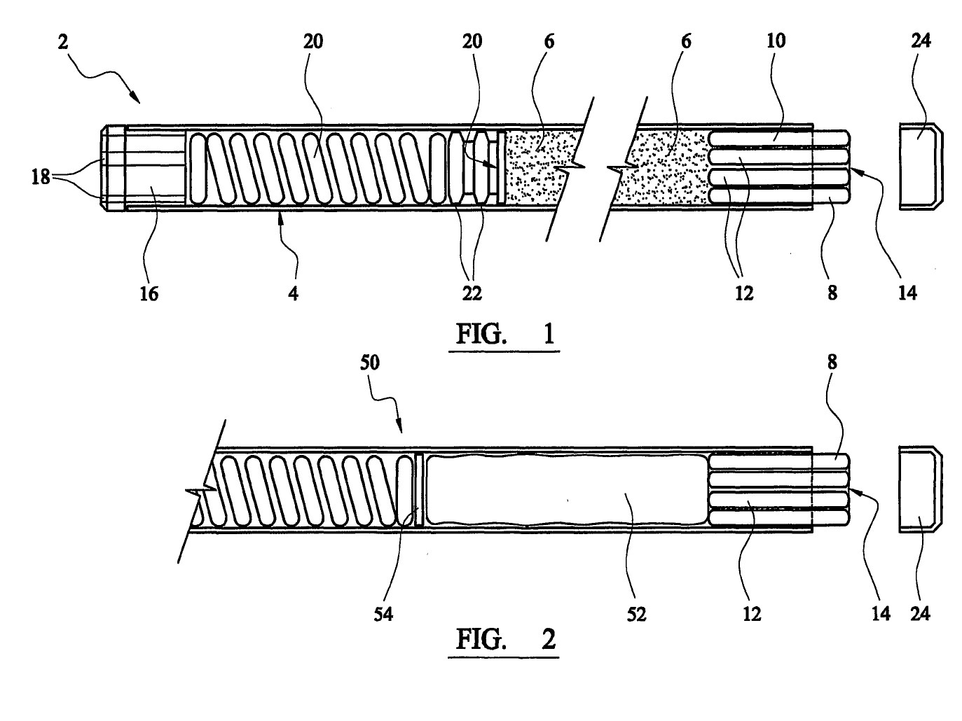

FIG. 1 is a side view, partially in section, through a drug delivery device according to the invention.

FIG. 2 is a side view, partially in section, through a second embodiment of drug delivery device.

FIGS. 3a and 3b are a side view through a third embodiment of a drug delivery device, before and after drug is discharged from the reservoir.

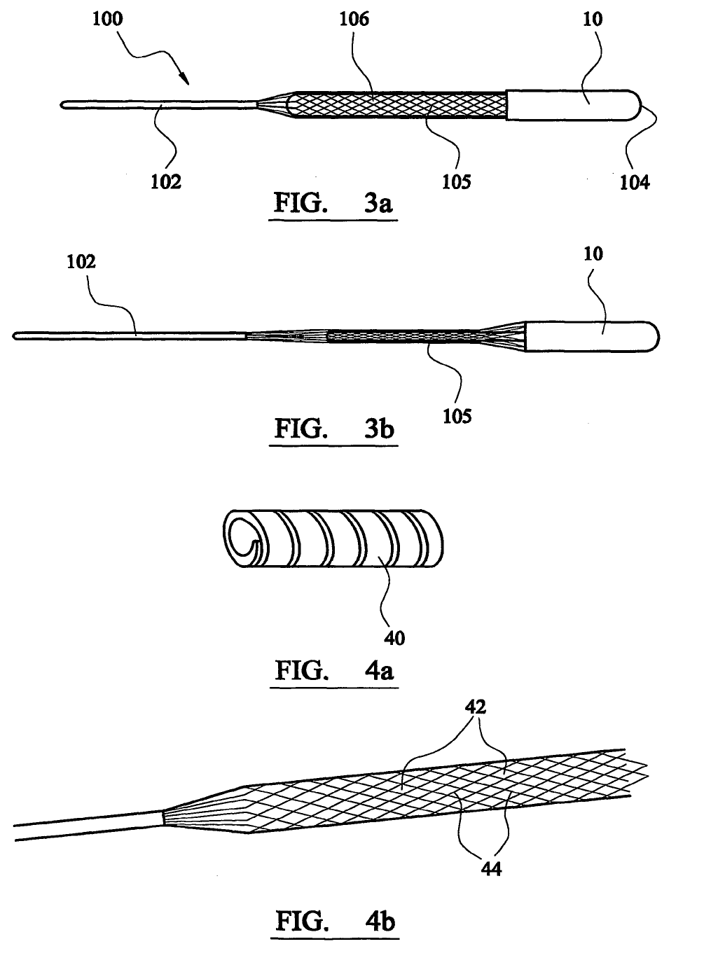

FIGS. 4a and 4b are isometric views of drive members which can be used in the device of the invention.

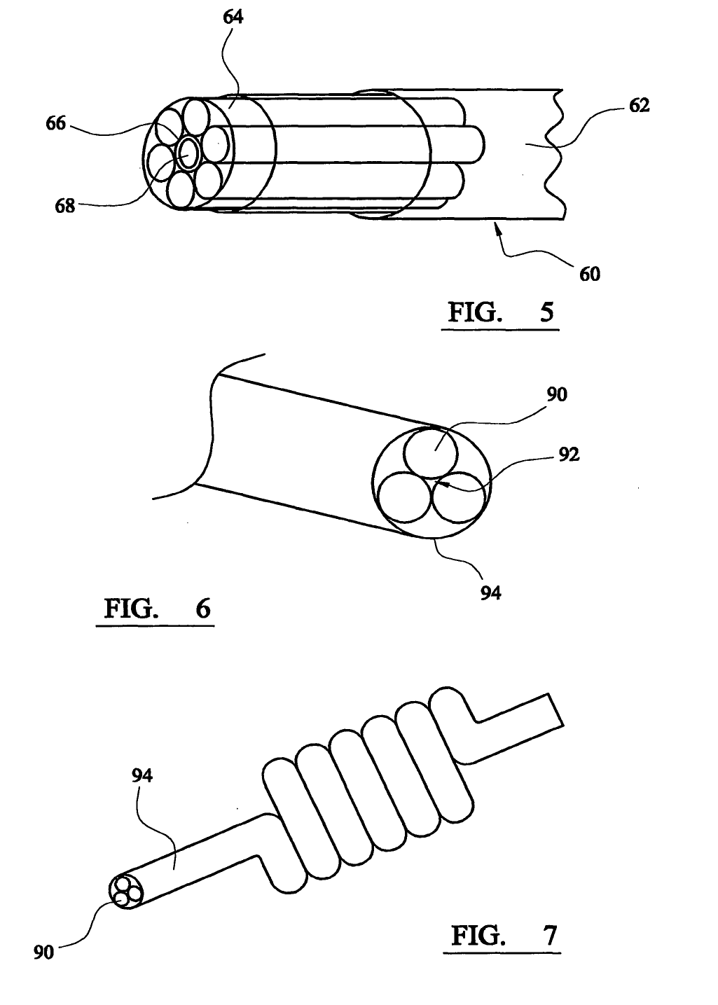

FIG. 5 is an enlarged isometric view of one end of the delivery device, showing an optional hollow tube rod member which allows the drug reservoir to be filled.

FIG. 6 is an isometric view of another embodiment of flow controller.

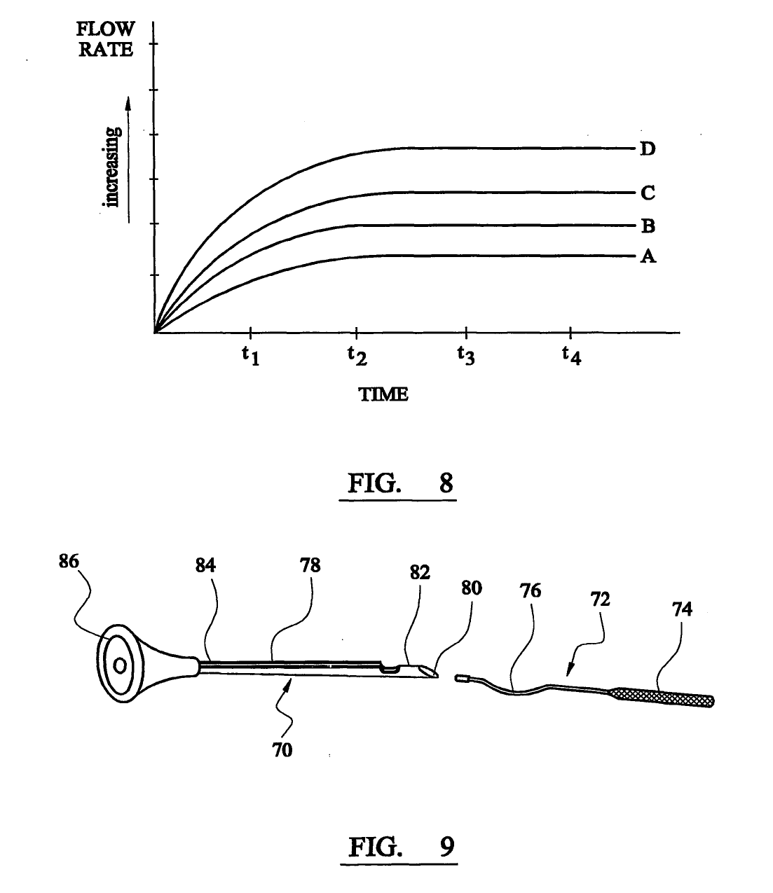

FIG. 7 is an isomeric view of yet another embodiment of flow controller.

FIG. 8 is a schematic view illustrating the effect of changing design parameters of the delivery device can affect the rate of flow of drug from the device.

FIG. 9 is a view of a drug delivery assembly which comprises an applicator and a delivery device which includes a retrieval line.

Referring to the drawings, FIG. 1 shows a drug delivery device 2 which comprises a hollow tubular housing 4 formed from a continuous tube of polytetrafluoroethylene whose wall is reinforced by helically wound fibres formed from, for example a polyester. The internal diameter of the tube is 1.0 mm and its wall thickness is about 0.1 mm. The overall length of the housing is about 35 mm.

The housing defines a reservoir 6 for the drug which is to be discharged. The drug is discharged from the reservoir at one end of the device through a discharge outlet 8. Flow of the drug through the discharge outlet is controlled by means of a flow controller 10. The flow controller comprises an array of seven rods which are arranged with six outer ones 12 of the rods arranged around a central rod 14 in a close packed array. The rods have substantially the same diameter so that each of the outer rods 12 is in contact with two neighbouring outer rods and with the central rod 14. Channels for drug to flow through the controller are defined between the rods, and between the rods and the internal wall of the tubular housing 4. The diameter of the rods is selected so that they can be force fitted into the tubular housing, where they are retained by frictional forces between the rods and the internal wall of the housing.

A closure plug 16 is provided at the end of the tubular housing that is opposite to the discharge outlet 8. The cap has vent openings 18 extending along its length so that fluid (air or a body fluid such as blood to which the device is exposed when in use) can flow into and out of the tubular housing at that end. A drive member 20 is located within the tubular housing, extending between from closure plug 16 towards the reservoir 6. The drive member is made from a shape memory alloy which consists of about 50.5 at-% nickel and about 49.5 at-% titanium. The alloy is treated using known techniques so that it exhibits enhanced elastic properties characterised by loading and unloading plateaus when deformed and subsequently relaxed, where changes in strain are accompanied by small changes in stress. The drive member is formed from a tube of the NiTi alloy by known techniques in which the tube is cut using a YAG laser. In the embodiment shown in FIG. 1, the drive member has a helical configuration.

A discharge piston 22 is located at the end of the drive member 20, between the drive member and the reservoir 6. The discharge piston includes a seal 22 around its periphery, to prevent liquid in the reservoir from flowing around the piston, out of the reservoir in a direction away from the discharge outlet 8. The seal is formed from a resiliently deformable material which is not affected adversely when contacted by the drug and with other fluids with which it comes into contact when in use.

A closure cap 24 covers the flow controller 10 at the discharge outlet end of the device, to prevent premature loss of drug from within the reservoir 6. The cap is formed from a material such as a gelatin which dissolves in body fluids when the device is implanted.

The device 2 is prepared for implantation by filling the reservoir 6 with the drug which is to be administered to a patient. It can be filled through one of the flow controller rods as described in more detail below. When the reservoir has been filled, the drive member 20 is compressed longitudinally so that it exerts a force on the reservoir in a direction towards the discharge outlet 8. Drug within the reservoir is prevented from being discharged through the discharge outlet by the presence of the closure cap 24 thereon.

The device can be implanted in a patient delivered to a desired location using known techniques which employ apparatus such as catheters, catheter guide wires, appropriate access ports and so on.

Once in a desired location, and once the closure cap has dissolved, drug is released from the reservoir 6 past the flow controller through the channels which are defined therein. The flow of drug from the reservoir is caused by action of the drive member on the discharge piston, which in turn acts on the reservoir. The force exerted by the drive member is substantially constant as it relaxes towards its undeformed configuration, corresponding to the stress on the “unloading plateau”. The rate at which drug is discharged from the device is determined by the force exerted by the drive member, the viscosity of the drug, the length, number and arrangement of the flow controller rods, and their surface finish.

FIG. 2 shows a drug delivery device 50 in which the reservoir is defined by a collapsible bladder 52 which is contained between the drive member 20 and the flow controller 10. The discharge piston 22 is provided by a cap on the end of the drive member 20.

In use, the delivery device is positioned in a desired location, for example using a catheter which the device can be fitted into as discussed above. Before being positioned in that location, it is filled with the drug that is to be administered to the patient. During the location procedure, the gelatin closure cap 24 is in place over the end of the flow controller 10. The reservoir 6 is under pressure from the drive member which is compressed between the reservoir and the closure plug 16 and the flow controller 10 with its closure cap 24. The closure cap dissolves in body fluids to which it is exposed after implantation. Once the flow controller, with its channels between the rods 12, 14 is open at its remote end to flow of drug, the drive member is able to move towards its undeformed configuration, causing drug to be displaced from the reservoir, out of the device through the channels in the flow controller.

FIG. 3a shows a delivery device 100 which is made from a shape memory alloy tube. The tube has a solid wall portion 102 which can be used as a retrieval line for the device. The tube contains a flow controller 10 at its remote end 104. Towards the remote end 104 of the tube, its wall is cut using a YAG laser to create a lattice 105 structure in which a network of limbs define apertures between them. The lattice structure enables the tube to be deformed outwardly so that its transverse dimension increases. This deformation involves bending of the limbs.

The device includes a reservoir 106 for a drug, which is located within the tube in the region in which it has been cut to give it the lattice structure. It will be appreciated however that a drive member with other constructions can be used with a reservoir in this way, including for example a drive member with a helical configuration such as is used in the devices shown in FIGS. 1 and 2.

Use of the device shown in FIG. 3 can involve similar procedural steps as those with the device of FIGS. 1 and 2. The supply of drug to the reservoir will involve deformation of the drive member to the configuration from which it recovers. In the case of the device shown in FIG. 3, the deformation involves increasing the transverse dimension of the drive member, involving bending deformation of the arms of the lattice structure, to the configuration shown in FIG. 3a. Subsequent discharge of drug from the reservoir through the discharge outlet involves inward collapse of the drive member and the reservoir, towards the configuration shown in FIG. 3b.

FIG. 4a shows a first embodiment of the drive member which consists of a tube of a NiTi shape memory alloy which has been treated with a YAG laser to cut a helical path so that the drive member consists of a helically wound filament 40. The drive member is annealed when in an elongated configuration to define the undeformed configuration of the member. The nature of the alloy and the treatment by which it has been prepared is such the member can be deformed by compressing it longitudinally (in the manner of a helical spring). This is the nature of the deformation to which the drive member is subjected when the delivery device is prepared for delivery of a drug. The drive member is able to recover from its deformed configuration towards the undeformed configuration as a result of the treatment to which the alloy of the drive member has been subjected.

The embodiment of drive member shown in FIG. 4b is again formed from a tube of a NiTi shape memory alloy which has been machined using a YAG laser. The drive member comprises a lattice structure in which apertures 42 are defined by a network of interconnected limbs 44. The configuration of the drive member can change to cause the discharge piston to move by bending the limbs, which causes the shape of the apertures to change.

FIG. 5 shows the end of a delivery device 60 comprising a tubular housing 62, which has a flow controller 64 extending from one end. The flow controller comprises a central rod with six outer rods arranged around it. The diameters of the central rod and of each of the outer rods is approximately the same. In the illustrated embodiment, the rods are straight. However, each of the outer rods can have a helical configuration so that they extend helically around the central rod. This has the advantage of increasing the length of the flow path defined by the rods.

In the illustrated embodiment, the central rod comprises a tube 66 which is open along its length so that drug can be supplied to the reservoir (not shown) within the tubular housing, for example using a syringe with an appropriate needle. The tube can be closed using a closure plug in the form of a length of wire 68 which is a tight fit in the tube 66. The central rod of the flow controller can have a solid cross-section.

FIG. 6 shows a flow controller which comprises three rods 90 arranged in a triangular array. A space 92 is defined between the rods by the surfaces of the rods. The space is generally triangular when the controller is viewed in cross-section, with each side of the triangle being concave. The controller includes an outer tube 94 which encloses the rods and holds them in a close packed array. The tube can be formed from a polymeric material or from a metal. It can be convenient for the tube to be made from a material which can be made to shrink when subjected to an appropriate treatment, especially on exposure to heat. For example, the outer tube of the flow controller can be made from a shape memory alloy. It can be made from a crosslinked polymer such as a crosslinked polyolefin such as a crosslinked polyethylene. Such materials can be made heat recoverable by forming them in a desired configuration (for example by extrusion in the case of a tubular product), crosslinking the material in that configuration, heating the material to a temperature which is above the softening point of the material, deforming the article to a configuration from which it is to recover (especially shrink), and restraining it in that configuration as it cools. On subsequent heating to a temperature above the softening point, without the restraint, the article will shrink or otherwise recover towards the original configuration in which it was formed.

FIG. 7 shows a flow controller which is formed from a construction of three rods 90 and an outer tube 94 as shown in FIG. 6. In the flow controller shown in FIG. 7, the entire construction of the rods and outer tube is formed into a helix, preferably with a constant diameter along a substantial part of its length. The generally triangular space 92 between the rods 90 will then adopt a helical shape. The flow path for a drug in the flow controller shown in FIG. 7 is longer than that for the flow controller shown in FIG. 6 (although for the same rod construction, the transverse dimension of the flow controller will be greater).

FIG. 8 is a graph which shows schematically how the rate of flow of drug through a flow controller (in particular, one which comprises a close-packed array of elongate rod members extending generally in the direction in which the drug flows out of the reservoir) can be varied by changing design parameters of the delivery device. The graph shows how flow rate can vary with time, depicting the flow rate behaviour for four different devices A, B, C and D, in which the flow rate increases from A to D. It shows how the flow rate increases from zero to a steady state level. The steady state flow rate can be obtained by using a drive member in which the force that is exerted by it does not vary significantly over a wide range of displacement which is conveniently obtained by using an appropriately treated shape memory alloy. The steady state flow rate depends on factors such as the force which is exerted on the reservoir by the drive member, the size of the channels in the flow controller, the length of the flow controller, the surface finish of the rod members of the flow controller.

FIG. 9 shows a drug delivery assembly which comprises an applicator 70 and a drug delivery device 72. The delivery device 72 includes a shape memory alloy drive member of the general kind discussed above. The drive member is formed at the end of a long tube 74 of the alloy material by laser cutting. The remaining portion 76 of the tube is left substantially uncut. It is however treated so that it exhibits enhanced elastic properties. The portion 76 of the tube can serve as a retrieval line, allowing the delivery device to be retrieved from the site at which the drug has been delivered, once sufficient drug has been delivered. The retrieval line can have to be passed along blood vessels whose tortuous shape means that the retrieval line is deformed as it passes along them. The enhanced elastic properties of the retrieval line (which might but need not necessarily be the same as the properties of the drive member) allow the retrieval line to be used in this way without being deformed permanently. Such permanent deformation could in some circumstances make it difficult for the retrieval line to pass along a blood vessel, for example when the delivery device is to be retrieved.

The applicator 70 comprises a hollow cannula 78 in which the delivery device can slide. It has a sharpened end 80 to facilitate penetration of the wall of a blood vessel. In a distal end region 82, the cannula has a closed cross-section. Between the distal end region and the proximal end 84, the cross-section is open. The retrieval line 76 can slide in the open section of the cannula. A grip 86 is provided at the proximal end by which the applicator can be manipulated.

1. A drug delivery device which comprises:
a. a reservoir for the drug, having a discharge outlet towards one end thereof through which the drug can be discharged,
b. a drive member which is arranged in the device to act against the reservoir directly or indirectly to cause the volume of the reservoir available for the drug to be reduced and to cause drug in the reservoir to be discharged from the reservoir at the end in which the discharge outlet is located,
c. a flow controller which controls flow of the drug through the discharge outlet, the flow controller comprising a close-packed array of elongate rod members extending generally in the direction in which the drug flows out of the reservoir.

2. A drug delivery device as claimed in claim 1, in which the rod members are straight and are arranged in the device so that they extend substantially parallel to the general direction in which the drug flows out of the reservoir.

3. A drug delivery device as claimed in claim 1, in which the flow controller comprises six outer rod members arranged around a central rod member.

4. A drug delivery device as claimed in claim 1, in which the rod members are retained in the reservoir by engaging the inner wall of the reservoir towards the end in which the discharge outlet is located.

5. A drug delivery device as claimed in claim 1, in which at least one of the rod members comprises a hollow tube and a plug which can be received in the tube to seal it against fluid flow.

6. A drug delivery device as claimed in claim 1, in which the drive member comprises a shape memory alloy which has been treated so that it exhibits enhanced elastic properties, which is arranged in the device in a compressed configuration to act against the reservoir directly or indirectly by recovering from its compressed configuration by virtue of its elastic properties to cause the volume of the reservoir available for the drug to be reduced and to cause drug in the reservoir to be discharged from the reservoir at the end in which the discharge outlet is located.

7. A drug delivery device as claimed in claim 1, in which the drive member positioned around the reservoir and recovers inwardly towards the reservoir from an expanded configuration.

8. A drug delivery device as claimed in claim 7, in which the drive member has a lattice structure comprising a network of interconnecting limbs defining apertures between them.

9. A drug delivery device as claimed in claim 1, in which the drive member has a helical configuration, at least in part.

10. A drug delivery device as claimed in claim 1, in which the drive member is located towards the end of the device that is opposite to the discharge outlet, the drive member recovering by expanding longitudinally from a compressed configuration.

11. A drug delivery device as claimed in claim 1, which includes a discharge piston which is acted on by the drive member, directly or indirectly, to cause it to move within the device towards the end in which the discharge outlet is located.

12. A drug delivery device as claimed in claim 11, which includes a hollow tube which contains the reservoir and the discharge piston which is arranged to slide therein.

13. A drug delivery device as claimed in claim 12, in which the drive member is contained within the hollow tube, and in which the device includes a retainer cap on the hollow tube at the end remote from the discharge outlet, for retaining the drive member within the tube.

14. A drug delivery device as claimed in claim 1, which includes a retainer cap at the end remote from the discharge outlet, which the drive member can act against.

15. A drug delivery device as claimed in claim 1, which includes a plug member to seal the reservoir at the end thereof against which the drive member acts.

16. A drug delivery device as claimed in claim 15, in which the plug member includes a gasket formed from a resilient material, in sliding contact with the wall of the reservoir.

17. A drug delivery device as claimed in claim 1, in which the reservoir includes a collapsible bladder for containing the drug.

18. A drug delivery device as claimed in claim 1, which includes a cap which can be fitted over the discharge outlet.

19. A drug delivery device as claimed in claim 1, in which the cap is made from a dissolvable material.

20. A drug delivery device as claimed in claim 1, which includes a retrieval line which is attached to the device and which can extend from the device towards an extraction site, and which can be used to apply force to the device to retrieve it from the location at which drug is discharged towards the extraction site.

21. A drug delivery assembly which comprises a device as claimed in claim 1, and an applicator for implanting the device in a body, the applicator comprising at least one of (a) a needle which has a bore extending at least part way along its length which is open at one end, in which the device can be fitted, and (b) a catheter having a bore extending at least part way along its length, in which the device can slide along the catheter.
